(19) United States
(12) Patent Application Publication
(10) Pub. No.: US 2020/0289015 A1
(43) Pub. Date: Sep. 17, 2020

(54) Multi-Retractable Electromyography Needle

(71) Applicant: Ryan Bowers, Lexington, SC (US)

(72) Inventor: Ryan Bowers, Lexington, SC (US)

(21) Appl. No.: 16/355,786

(22) Filed: Mar. 17, 2019

(51) Int. Cl. A61B 5/0492

(52) U.S. Cl. CPC A61B 5/0492; A61B 5/6848

(57) ABSTRACT

The invention disclosed in this application is an electromyography needle that contains smaller internal electrodes. When inserted into a patient, the smaller electrodes are deployed from the main carrier needle into the surrounding muscle or tissue. The deployment of the multiple electrodes from a single needle increases the surface area for the electromyography test and reduces patient discomfort.

### CROSS REFERENCE TO RELATED APPLICATIONS

Not Applicable

### STATEMENT REGARDING FEDERALLY SPONSORED RESEARCH OR DEVELOPMENT

This invention was not made by an agency of the United States Government nor under a contract with an agency of the United States Government.

### THE NAME OF THE PARTIES TO JOINT RESEARCH AGREEMENT

Not Applicable.

### INCORPORATION BY REFERENCE OF MATERIAL SUBMITTED ON A COMPACT DISC OR AS A TEXT FILE VIA THE OFFICE ELECTRONIC FILING SYSTEM (EFS-WEB)

Not Applicable.

### STATEMENT REGARDING PRIOR DISCLOSURES BY THE INVENTOR OR A JOINT INVENTOR

Not Applicable

### BACKGROUND OF THE INVENTION

### Field of the Invention

Electromyography (EMG) is used for recording and evaluating the electrical activity produced by skeletal muscles. EMG is used as a diagnostic tool for the identification of neuromuscular diseases or as a research tool. There are two types of EMG: surface and intramuscular.

Surface EMG assesses muscle function by recording the activity of the muscle from the skin above the muscle and provides a limited assessment of muscle activity as it is restricted to use with superficial muscles.

Intramuscular EMG involves the insertion of an electrode under the skin into the muscle for recording the electrical activity of the muscle. It is to this type of EMG that the present invention is directed.

### Description of Related Art

U.S. Pat. No. 7,283,866 (Needle Having Multiple Electrodes, issued Oct. 16, 2007 to Mumford, et al.) describes a needle for use with EMG where the needle has three electrodes. These electrodes are stationary with respect to the needle and is thus significantly different from the present invention, wherein multiple electrodes are deployed away from the needle into the surrounding muscle.

### BRIEF SUMMARY OF THE INVENTION

The present invention relates to an electromyography needle that contains smaller internal electrodes. When inserted into a patient, the smaller electrodes are deployed from the main carrier needle into the surrounding muscle or tissue. The deployment of the multiple electrodes from a single needle increases the surface area for the EMG test and reduces patient discomfort.

### BRIEF DESCRIPTION OF THE SEVERAL VIEWS OF THE DRAWING

FIG. 1 is a view of a needle (10) with three electrodes (20) which are have not been deployed by pressing the plunger (30) towards the tip of the needle (11).

FIG. 2 is a view of a needle (10) with three electrodes (20) which have been deployed from the interior of the needle (10) through the needle's apertures.

FIG. 3 is a cross-section view of the needle (10) with three electrodes (20).

FIG. 4 is a cross-section view of the needle (10) with four electrodes (20).

FIG. 5 is a cross-section view of the needle (10) with five electrodes (20).

FIG. 6 is a cross-section view of the needle (10) with seven electrodes (20).

FIG. 7 is a cross-section view of the needle (10) with fourteen electrodes (20).

FIG. 8 is a side view of the needle (10) showing the aperture (12) in the tip (11) with the end of the electrode (20) seen through the aperture (12).

FIG. 9 is a side cross-section view of the needle (10) showing the aperture (12) in the tip (11) with the electrode (20) inside the needle (10).

FIG. 10 is a top view of the needle tip (11) showing the aperture (12) and the end of the electrodes (20).

FIG. 12 is a depiction of the needle (10) inserted into a patient and the electrodes (20) deployed.

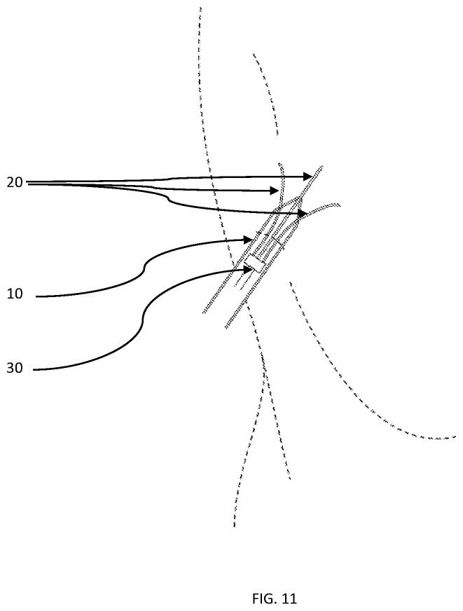

### DETAILED DESCRIPTION OF THE INVENTION

Electromyography (EMG) is used for recording and evaluating the electrical activity produced by skeletal muscles. EMG is used as a diagnostic tool for the identification of neuromuscular diseases or as a research tool. Intramuscular EMG involves the insertion of an electrode under the skin into the muscle for recording the electrical activity of the muscle. It is to this type of EMG that the present invention is directed.

The present invention, Multi-Retractable Electromyography Needle is an electromyography needle that contains smaller internal electrodes. When inserted into a patient, the smaller electrodes (20) are deployed from the main carrier needle (10) into the surrounding muscle. The deployment of the multiple electrodes (20) from a single needle (10) increases the surface area for the EMG test and reduces patient discomfort.

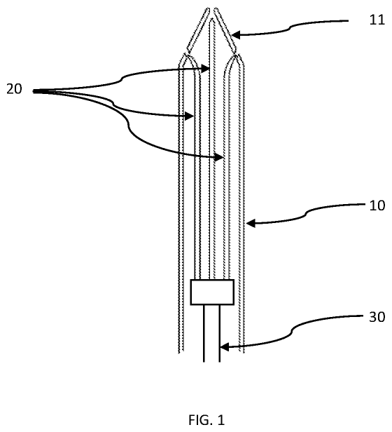

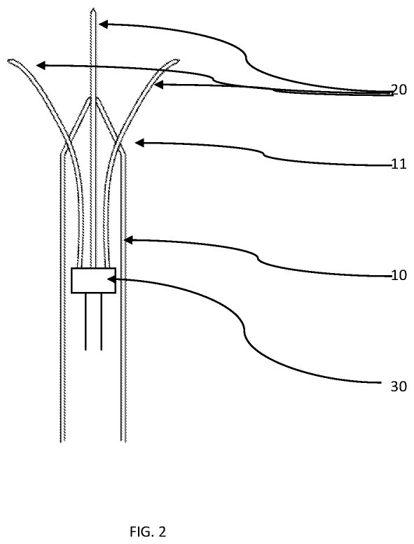

As shown in FIG. 1, the carrier needle (10) is equipped with multiple exit apertures at the tip (11) of the needle. Inside the carrier needle (10) are a number of smaller electrodes (20) which are deployed through the apertures at the tip (11) of the needle (10) when the plunger (30) is moved towards the tip (11) of the needle as shown in FIG. 2.

When the plunger (30) is moved away from the carrier needle's tip (11), the electrodes (20) are pulled back through the carrier needle's apertures (12) into the interior of the carrier needle (10).

There are a number of ways that the plunger (30) could be moved to deploy and retract the electrodes (20). One method would be a device similar to the syringe used for medication injections via a hypodermic needle. Another would be a spring-loaded device of the type used with a retractable writing pen.

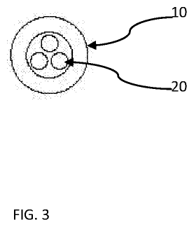

The carrier needle (10) may contain between three and fourteen electrodes (20) depending upon the size of the carrier needle. Although it should be recognized that the number of electrodes (20) is not limited to fourteen, larger carrier needles (10) could contain more than fourteen electrodes. The invention embodiments with three to fourteen electrodes are depicted in FIG. 3 through FIG. 7.

FIG. 3 is a cross-section view of a carrier needle (10) with three electrodes (20). In this configuration, the carrier needle (10) would be a size 21 gauge with size 38 gauge electrodes (20). A size 21 gauge needle has an outer diameter range of 0.81 to 0.83 millimeters (mm) with an inner diameter range of 0.50 to 0.53 mm. A size 38 gauge wire has a nominal outer diameter of 0.10 mm.

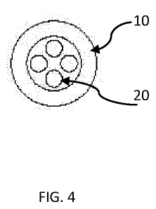

FIG. 4 is a cross-section view of a carrier needle (10) with four electrodes (20). In this configuration, the carrier needle (10) would be a size 20 gauge with size 38 gauge electrodes (20). A size 20 gauge needle has an outer diameter range of 0.90 to 0.91 millimeters (mm) with an inner diameter range of 0.58 to 0.62 mm.

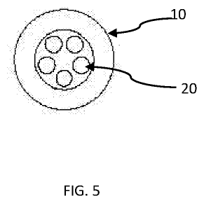

FIG. 5 is a cross-section view of a carrier needle (10) with five electrodes (20). In this configuration, the carrier needle (10) would be a size 19 gauge with size 38 gauge electrodes (20). A size 19 gauge needle has an outer diameter range of 1.05 to 1.08 millimeters (mm) with an inner diameter range of 0.65 to 0.72 mm.

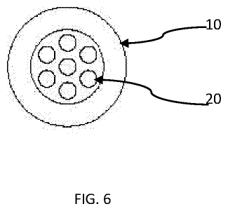

FIG. 6 is a cross-section view of a carrier needle (10) with seven electrodes (20). In this configuration, the carrier needle (10) would be a size 18 gauge with size 38 gauge electrodes (20). A size 18 gauge needle has an outer diameter range of 1.26 to 1.28 millimeters (mm) with an inner diameter range of 0.80 to 0.88 mm.

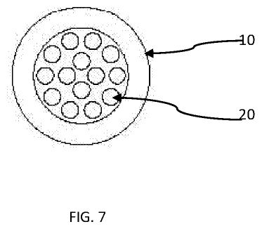

FIG. 7 is a cross-section view of a carrier needle (10) with fourteen electrodes (20). In this configuration, the carrier needle (10) would be a size 17 gauge with size 38 gauge electrodes (20). A size 17 gauge needle has an outer diameter range of 1.46 to 1.49 millimeters (mm) with an inner diameter range of 1.03 to 1.10 mm.

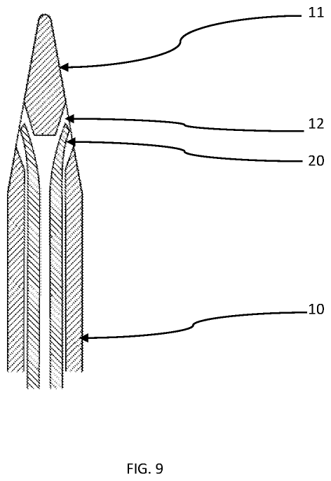

As seen in FIG. 2 and FIG. 9, the ends of the electrodes (20) are slightly curved to assist in the deployment of the electrode (20) away from the carrier needle (10) and into the muscle tissue.

The tip (11) of the needle (10) is equipped with a number of apertures (12) to allow passage of the electrodes (20) from the interior of the needle (10) to the outside of the needle (10). This is for the deployment of the electrodes (20) into the surrounding tissue once the needle (10) has been inserted into the patient's body.

The preferred embodiment of the carrier needle (10) is constructed of medical grade stainless steel with the exterior coated in Teflon, excepting the tip. Construction of the needle (10) is not limited to medical grade stainless steel as other metals could be used, including tungsten, platinum, copper or silver-silver chloride alloy. The preferred embodiment of the electrodes (20) are constructed of copper, but other metals could be used including silver, gold, aluminum, zinc and nickel.

Although the FIGS. 3 through 7 represent the preferred embodiments with respect to needle size and number of wires, it should be recognized that the invention is not limited to the sizes described in paragraphs 028 through 033.

Potential CPC patent classifications for this invention include:
Class A61: Medical or Veterinary Sciences; Hygiene
Subclass A616: Diagnosis; Surgery; Identification
Subclass A61N: Electrotherapy; Magneotherapy; Radiation Therapy; Ultrasound Therapy

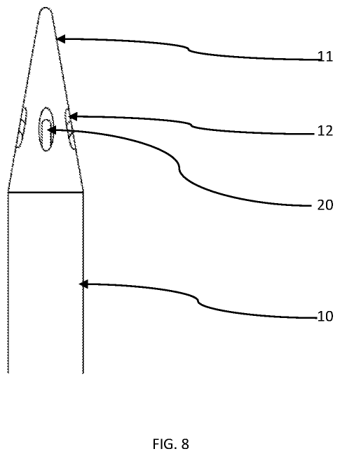

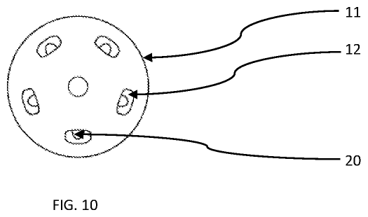

The present invention described above and illustrated in FIGS. 1 through 11 is visualized as the preferred embodiment of the invention. It is envisioned that this invention may be embodied in many different forms and should not be construed as limited to the embodiments set forth herein. It will be understood by those skilled in the art that changes in forms and details may be made without departing from the spirit and scope of the present application. It is therefore intended that the present invention not be limited to the exact forms and details described and illustrated herein but falls within the scope of the appended claims.

The terminology used herein is for describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a stricture or feature that is disposed “adjacent” another feature may have portions that overlap or underlie the adjacent feature.

The invention claimed is:

1. A Multi-Retractable Electromyography Needle comprising an outer needle containing between three and fourteen electromyography electrodes where said electrodes can be deployed from and retracted into the outer needle through apertures in the needle tip.

2. The needle of claim 1, wherein an outer diameter of the needle is between 0.8 to 1.5 millimeters.

3. The needle of claim 1, wherein an inner diameter of the needle is between 0.5 to 1.1 millimeters.

4. The needle of claim 1, wherein the needle comprises a biocompatible material selected from the group consisting of: stainless steel, tungsten, platinum, silver-silver chloride alloy and copper.

5. The electrode of claim 1, wherein the electrode is 38 gauge.

6. The electrode of claim 1, where the electrode comprises a biocompatible material selected from the group consisting of: copper, gold, silver, aluminum, zinc and nickel.

7. The needle of claim 1, wherein the needle tip is equipped with apertures ranging in number from three to fourteen to allow for deployment of the electrodes from the interior of the needle.

8. The needle of claim 1, wherein the exterior of the needle excepting the tip is coated in Teflon.
